(19) United States
(12) Patent Application Publication
(10) Pub. No.: US 2008/0020998 A1
(43) Pub. Date: Jan. 24, 2008

(54) Sustained release compositions for the therapeutic management of pain, inflammation and inflammation-associated disorders and prophylactic/ therapeutic methods thereof

(76) Inventor: Muhammed Majeed, Piscataway, NJ (US)

Correspondence Address: SABINSA CORPORATION, 70 ETHEL ROAD WEST, UNIT 6, PISCATAWAY, NJ, 08854, US

(21) Appl. No.: 11/782,044

(22) Filed: Jul. 24, 2007

Related U.S. Application Data
(60) Provisional application No. 60/767,557, filed on Jul. 24, 2006.

(51) Int. Cl. A61K 31/70; A61K 31/12; A61K 31/19; A61P 29/00; A61P 19/00; A61K 31/445

(52) U.S. Cl. 514/62; 514/315; 514/569; 514/679

(57) ABSTRACT

Disclosed are sustained-release medicaments/drug compositions/formulations/therapeutics for the management of pain and inflammation associated disorders in general, more particularly arthritic inflammation. The sustained-release compositions comprise effective amounts of glucosamine, curcuminoids, boswellic acids and piperine.

This application is a non-provisional application of provisional application US 60/767,557 filed on Jul. 24, 2006.

### BACKGROUND OF THE INVENTION

1. Field of the Invention

The present invention relates to sustained-release medicaments/drug compositions/formulations/therapeutics for the management of pain and inflammation associated disorders in general. More specifically, the sustained release compositions of the present invention comprise effective amounts of glucosamine, curcuminoids, boswellic acids and piperine.

2. Description of Prior Art

Inflammation is one of the body's natural reactions to disease or injury, and includes swelling, pain, and stiffness. Inflammation that lasts for a very long time or recurs, as in arthritis, can lead to tissue damage. The word “arthritis” means “joint inflammation. (“Arth” means joint, “itis” means inflammation).

More than 100 forms of arthritis and related diseases have been identified to affect approximately 46 million Americans today. The list includes Achilles tendonitis, Achondroplasia, Acromegalic arthropathy, Adhesive capsulitis, Adult onset Still's disease, Ankylosing spondylitis, Anserine bursitis, Avascular necrosis, Behcet's syndrome, Bicipital tendonitis, Blount's disease, Brucellar spondylitis, Bursitis, Calcaneal bursitis, Calcium pyrophosphate dihydrate (CPPD), Crystal deposition disease, Caplan's syndrome, Carpal tunnel syndrome, Chondrocalcinosis, Chondromalacia patellae, Chronic synovitis, Chronic recurrent multifocal osteomyelitis, Churg-Strauss syndrome, Cogan's syndrome, Corticosteroid-induced osteoporosis, Costosternal syndrome, CREST syndrome, Cryoglobulinemia, Degenerative joint disease, Dermatomyositis, Diabetic finger sclerosis, Diffuse idiopathic skeletal hyperostosis (DISH), Discitis, Discoid lupus erythematosus, Drug-induced lupus, Duchenne's muscular dystrophy, Dupuytren's contracture, Ehlers-Danlos syndrome, Enteropathic arthritis, Epicondylitis, Erosive inflammatory osteoarthritis, Exercise-induced compartment syndrome, Fabry's disease, Familial Mediterranean fever, Farber's lipogranulomatosis, Felty's syndrome, Fibromyalgia, Fifth's disease, Flat feet, Foreign body synovitis, Freiberg's disease, Fungal arthritis, Gaucher's disease, Giant cell arthritis, Gonococcal arthritis, Goodpasture's syndrome, Gout, Granulomatous arthritis, Hemarthrosis, hemochromatosis, Henoch-Schonlein purpura, Hepatitis B surface antigen disease, Hip dysplasia, Hurler syndrome, Hypermobility syndrome, Hypersensitivity vasculitis, Hypertrophic osteoarthropathy, Immune complex disease, Impingement syndrome, Jaccoud's arthropathy, Juvenile ankylosing spondylitis, Juvenile dermatomyositis, Juvenile rheumatoid arthritis, Kawasaki disease, Kienbock's disease, Legg-Calve-Perthes disease, Lesch-Nyhan syndrome, Linear scleroderma, Lipoid dermatoarthritis, Lofgren's syndrome, Lyme disease, Malignant synovioma, Marfan's syndrome, Medial plica syndrome, Metastatic carcinomatous arthritis, Mixed connective tissue disease (MCTD), Mixed cryoglobulinemia, Mucopolysaccharidosis, Multicentric reticulo-histiocytosis, Multiple epiphyseal dysplasia, Mycoplasmal arthritis, Myofascial pain syndrome, Neonatal lupus, Neuropathic arthropathy, Nodular panniculitis, Ochronosis, Olecranon bursitis, Osgood-Schlatter's disease, Osteoarthritis, Osteochondromatosis, Osteogenesis imperfecta, Osteomalacia, Osteomyelitis, Osteonecrosis, Osteoporosis, Overlap syndrome, Pachydermoperiostosis Paget's disease of bone, Palindromic rheumatism, Patellofemoral pain syndrome, Pellegrini-Stieda syndrome, Pigmented villonodular synovitis, Piriformis syndrome, Plantar fasciitis, Polyarteritis nodosa, Polymyalgia rheumatica, Polymyositis, Popliteal cysts, Posterior tibial tendonitis, Pott's disease, Prepatellar bursitis, Prosthetic joint infection, Pseudoxanthoma elasticum, Psoriatic arthritis, Raynaud's phenomenon, Reactive arthritis/Reiter's syndrome, Reflex sympathetic dystrophy syndrome, Relapsing polychondritis, Retrocalcaneal bursitis, Rheumatic fever, Rheumatoid arthritis, Rheumatoid vasculitis, Rotator cuff tendonitis, Sacroiliitis, Salmonella osteomyelitis, Sarcoidosis, Saturnine gout, Scheuermann's osteochondritis, Scleroderma, Septic arthritis, Seronegative arthritis, Shigella arthritis, Shoulder-hand syndrome, Sickle cell arthropathy, Sjogren's syndrome, Slipped capital femoral epiphysis, Spinal stenosis, Spondylolysis, Staphylococcus arthritis, Stickler syndrome, Sub-acute cutaneous lupus, Sweet's syndrome, Sydenham's chorea, Syphilitic arthritis, Systemic lupus erythematosus (SLE), Takayasu's arteritis, Tarsal tunnel syndrome, Tennis elbow, Tietse's syndrome, Transient osteoporosis, Traumatic arthritis, Trochanteric bursitis, Tuberculosis arthritis, Arthritis of Ulcerative colitis, Undifferentiated connective tissue syndrome (UCTS), Urticarial vasculitis, Viral arthritis, Wegener's granulomatosis, Whipple's disease, Wilson's disease and Yersinial arthritis.

Different types of arthritis have different symptoms and the symptoms vary in severity from person to person. Symptoms of other types of arthritis may include fatigue, fever, a rash and the signs of joint inflammation, including, (i) Pain, (ii) Swelling, (iii) Stiffness, (iv) Tenderness, (v) Redness and (vi) Warmth.

The inflammatory process in arthritis correlates strongly with the balance of pro- and anti-inflammatory cytokine expression in the joints. (Clinical & Experimental Immunology, Volume 146, Number 2, November 2006, pp. 287-293(7)). Pro-inflammatory cytokines also play a decisive role in the generation of inflammatory and destructive responses in rheumatoid arthritis. (Clinical Reviews in Allergy and Immunology, Volume 28, Number 3, June 2005, pp. 239-248(10)).

The leukotrienes are yet another family of pro-inflammatory lipid mediators implicated in arthritic inflammation which are derived from arachidonic acid through the action of 5-lipoxygenase (5-LOX). There are 2 major families of leukotrienes: (1) that of leukotriene B4 (LTBR4) and (2) the 3-cysteinyl leukotrienes, which include LTC4, LTD4, and LTE4. Both families of leukotrienes exert a wide spectrum of biologic effects involved in the initiation and perpetuation of inflammation. (Simms R W, Korn J H. The role of leukotrienes in alveolitis associated with scleroderma. Arthritis Rheum. 2003, 48:1478-1480). Marked elevation of both leukotriene B4 (LTB4) and the cysteinyl leukotrienes in diseased joints has been documented earlier. (Klickstein, L. B., C. Shapleigh, and E. J. Goetzl. 1980. Lipoxygenation of arachidonic acid as a source of polymorphonuclear leukocyte chemotactic factors in synovial fluid and tissue in rheumatoid arthritis and spondyloarthritis. J. Clin. Invest. 66:1166-1170 and Koshihara, Y., T. Isono, H. Oda, S. Karube, and Y. Hayashi. 1988. Measurement of sulfidopeptide leukotrienes and their metabolism in human synovial fluid of patients with rheumatoid arthritis. Prostaglandins Leukot. Essent. Fatty Acids. 32:113-119). The requirement of neutrophil derived leukotriene B4 for inflammatory arthritis has been documented in J Exp Med. 2006 Apr. 17; 203(4):837-42. Epub 2006 Mar. 27.

The role of prostaglandins as pro-inflammatory mediators has been documented in J Clin Invest. 1992 January, 89(1): 97-108. The shunt from the cyclooxygenase to lipoxygenase pathway in human osteoarthritis has been documented as Arthritis Research & Therapy 2006, 8:R181 doi: 10.1186/ar2092.

Pro inflammatory cytokines also stimulate matrix metalloproteinase enzymes that they destroy the extracellular matrix exacerbating the inflammation caused due to arthritis. The concept that cytokine (interleukin 1-beta and tumor necrosis 1 alpha)-matrix metalloproteinases (MMP) associations reflect a modified chondrocyte phenotype and an intrinsic process of cartilage degradation in OA has been documented in Arthritis Rheum. 2001 March, 44(3):585-94. The stimulation of MMP-I by IL-1β and TNF-α has been documented in Pancreatology 2003, Vol 3, No. 5 2003:414-421.

Thus, the therapeutic management of inflammatory arthritis calls for the effectively countering (i) the deleterious activities of pro inflammatory cytokines in degrading the matrix metalloproteinases, (ii) the inhibition of leukotriene B4 synthesis by 5-lipoxygenase enzyme, and (iii) production of prostaglandins by cyclooxygenase enzymes.

The use of plant based ingredients as a medication for the therapeutic management of pain and inflammation associated disorders, in particular inflammatory arthritis is known in the art.

US2004121024, titled “Composition for the treatment and prevention of osteoarthritis, rheumatoid arthritis and improved joint function” by GORSEK WAYNE F on Jun. 24, 2004 discusses a composition for oral ingestion that contains effective amounts of Glucosamine sulfate, Nettle Leaf, Quercetin, curcumin extract/curcuminoids, Selenium, Zinc, Vitamin C (calcium ascorbate) and Grape Seed Extract, as well as other ingredients and healthy filler ingredients.

US2006280811, titled “Formulations for the treatment of arthritis conditions”, by Bombardelli Ezio on Dec. 14, 2006 discusses formulations comprising combinations of analgesic/anti-inflammatory, immunomodulating and cartilage-reconstructing agents in particular comprising saligenig, boswellic acid, procyanidins, N-acety-glucosamine and either glucoronic acid or glucoronolactone, for the treatment of rheumatoid arthritis and, more generally, of arthritis conditions.

However, in general the prior art references suffer from one or more disadvantages like,
1. The lack of a comprehensive, therapeutic regimen for countering the arthritic inflammation caused by (i) The induction of matrix metallo-proteinases by the pro inflammatory cytokines, (ii) Prostaglandin synthesis by the action of cyclo oxygenase enzymes, (iii) Leukotriene B4 synthesis by 5-lipoxygenase enzyme, and (iv) Breakdown of the connective tissue framework.
2. The lack of a comprehensive, therapeutic regimen for countering the arthritic inflammation which takes care of the issues of bioavailability of glucosamine, curcuminoids and boswellic acids. The half life (t^{1/2}) of curcumin and boswellic acids is relatively short, making it very difficult to maintain the minimum efficacious levels in the blood to reap maximum therapeutic benefits.
3. The lack of a comprehensive, therapeutic regimen capable of bringing about an exponential decrease in the levels of pro inflammatory mediators implicated in inflammatory arthritis.

It is thus the principle object of the present invention to develop a comprehensive therapeutic regimen for countering pain, inflammation, inflammation associated disorders in particular, arthritic inflammation caused by (i) The induction of matrix metallo-proteinases by the pro inflammatory cytokines, (ii) Prostaglandin synthesis by the action of cyclo oxygenase enzymes, (iii) Leukotriene B4 synthesis by 5-lipoxygenase enzyme, and (iv) Breakdown of the connective tissue framework.

It is also an object of the present invention to develop a comprehensive, therapeutic regimen for countering the arthritic inflammation which takes care of the issues of bioavailability of glucosamine, curcuminoids and boswellic acids.

It is further an object of the present invention to develop a comprehensive therapeutic regimen that would bring about an exponential decrease in the levels of pro inflammatory mediators that are implicated in inflammatory arthritis.

It is yet another object of the present invention to develop a comprehensive therapeutic regimen for countering the various types of arthritic inflammation including Achilles tendonitis, Achondroplasia, Acromegalic arthropathy, Adhesive capsulitis, Adult onset Still's disease, Ankylosing spondylitis, Anserine bursitis, Avascular necrosis, Behcet's syndrome, Bicipital tendonitis, Blount's disease, Brucellar spondylitis, Bursitis, Calcaneal bursitis, Calcium pyrophosphate dihydrate (CPPD), Crystal deposition disease, Caplan's syndrome, Carpal tunnel syndrome, Chondrocalcinosis, Chondromalacia patellae, Chronic synovitis, Chronic recurrent multifocal osteomyelitis, Churg-Strauss syndrome, Cogan's syndrome, Corticosteroid-induced osteoporosis, Costosternal syndrome, CREST syndrome, Cryoglobulinemia, Degenerative joint disease, Dermatomyositis, Diabetic finger sclerosis, Diffuse idiopathic skeletal hyperostosis (DISH), Discitis, Discoid lupus erythematosus, Drug-induced lupus, Duchenne's muscular dystrophy, Dupuytren's contracture, Ehlers-Danlos syndrome, Enteropathic arthritis, Epicondylitis, Erosive inflammatory osteoarthritis, Exercise-induced compartment syndrome, Fabry's disease, Familial Mediterranean fever, Farber's lipogranulomatosis, Felty's syndrome, Fibromyalgia, Fifth's disease, Flat feet, Foreign body synovitis, Freiberg's disease, Fungal arthritis, Gaucher's disease, Giant cell arthritis, Gonococcal arthritis, Goodpasture's syndrome, Gout, Granulomatous arthritis, Hemarthrosis, hemochromatosis, Henoch-Schonlein purpura, Hepatitis B surface antigen disease, Hip dysplasia, Hurler syndrome, Hypermobility syndrome, Hypersensitivity vasculitis, Hypertrophic osteoarthropathy, Immune complex disease, Impingement syndrome, Jaccoud's arthropathy, Juvenile ankylosing spondylitis, Juvenile dermatomyositis, Juvenile rheumatoid arthritis, Kawasaki disease, Kienbock's disease, Legg-Calve-Perthes disease, Lesch-Nyhan syndrome, Linear scleroderma, Lipoid dermatoarthritis, Lofgren's syndrome, Lyme disease, Malignant synovioma, Marfan's syndrome, Medial plica syndrome, Metastatic carcinomatous arthritis, Mixed connective tissue disease (MCTD), Mixed cryoglobulinemia, Mucopolysaccharidosis, Multicentric reticulohistiocytosis, Multiple epiphyseal dysplasia, Mycoplasmal arthritis, Myofascial pain syndrome, Neonatal lupus, Neuropathic arthropathy, Nodular panniculitis, Ochronosis, Olecranon bursitis, Osgood-Schlatter's disease, Osteoarthritis, Osteochondromatosis, Osteogenesis imperfecta, Osteomalacia, Osteomyelitis, Osteonecrosis, Osteoporosis, Overlap syndrome, Pachydermoperiostosis Paget's disease of bone, Palindromic rheumatism, Patellofemoral pain syndrome, Pellegrini-Stieda syndrome, Pigmented villonodular synovitis, Piriformis syndrome, Plantar fasciitis, Polyarteritis nodosa, Polymyalgia rheumatica, Polymyositis, Popliteal cysts, Posterior tibial tendonitis, Pott's disease, Prepatellar bursitis, Prosthetic joint infection, Pseudoxanthoma elasticum, Psoriatic arthritis, Raynaud's phenomenon, Reactive arthritis/Reiter's syndrome, Reflex sympathetic dystrophy syndrome, Relapsing polychondritis, Retrocalcaneal bursitis, Rheumatic fever, Rheumatoid arthritis, Rheumatoid vasculitis, Rotator cuff tendonitis, Sacroiliitis, Salmonella osteomyelitis, Sarcoidosis, Saturnine gout, Scheuermann's osteochondritis, Scleroderma, Septic arthritis, Seronegative arthritis, Shigella arthritis, Shoulder-hand syndrome, Sickle cell arthropathy, Sjogren's syndrome, Slipped capital femoral epiphysis, Spinal stenosis, Spondylolysis, Staphylococcus arthritis, Stickler syndrome, Sub-acute cutaneous lupus, Sweet's syndrome, Sydenham's chorea, Syphilitic arthritis, Systemic lupus erythematosus (SLE), Takayasu's arteritis, Tarsal tunnel syndrome, Tennis elbow, Tietse's syndrome, Transient osteoporosis, Traumatic arthritis, Trochanteric bursitis, Tuberculosis arthritis, Arthritis of Ulcerative colitis, Undifferentiated connective tissue syndrome (UCTS), Urticarial vasculitis, Viral arthritis, Wegener's granulomatosis, Whipple's disease, Wilson's disease and Yersinial arthritis which are characterized by (i) The induction of matrix metallo-proteinases by the pro inflammatory cytokines, (ii) Prostaglandin synthesis by the action of cyclo oxygenase enzymes, (iii) Leukotriene B4 synthesis by 5-lipoxygenase enzyme, and (iv) Breakdown of the connective tissue framework.

The present invention fulfills these objectives and provides further related advantages.

### SUMMARY OF THE INVENTION

The present invention describes a comprehensive sustained-release medicaments/drug compositions/formulations/therapeutics for the management of pain and inflammation associated disorders, in particular the prevention and treatment of the entire gamut of arthritic inflammations. The sustained-release compositions of the present invention exert their action by (i) inhibiting the action of matrix-metalloproteinases by pro inflammatory enzymes; (ii) Inhibiting prostaglandin synthesis by the action of cyclo oxygenase enzymes, (iii) Inhibiting Leukotriene B4 synthesis by 5-lipoxygenase enzyme; and (iv) rebuilding of the connective tissue framework. The sustained-release compositions ensure that effective amounts of actives included therein like glucosamine, curcuminoids, boswellic acids and piperine are achieved in the blood over a prolonged period of time to achieve maximum therapeutic benefits.

The current invention presents the following advantageous features.
(A) A comprehensive therapeutic regimen for countering pain, inflammation, inflammation-associated disorders in particular, arthritic inflammation caused by (i) The induction of matrix metallo-proteinases by the pro inflammatory cytokines, (ii) Prostaglandin synthesis by the action of cyclo oxygenase enzymes, (iii) Leukotriene B4 synthesis by 5-lipoxygenase enzyme, and (iv) Breakdown of the connective tissue framework.
(B) A comprehensive, sustained-release therapeutic regimen for countering the arthritic inflammation which takes care of the issues of bioavailability of glucosamine, curcuminoids and boswellic acids ensuring efficacious amounts of the said actives over a prolonged period of time.
(C) A comprehensive therapeutic regimen for countering the various types of arthritic inflammation including Achilles tendonitis, Achondroplasia, Acromegalic arthropathy, Adhesive capsulitis, Adult onset Still's disease, Ankylosing spondylitis, Anserine bursitis, Avascular necrosis, Behcet's syndrome, Bicipital tendonitis, Blount's disease, Brucellar spondylitis, Bursitis, Calcaneal bursitis, Calcium pyrophosphate dihydrate (CPPD), Crystal deposition disease, Caplan's syndrome, Carpal tunnel syndrome, Chondrocalcinosis, Chondromalacia patellae, Chronic synovitis, Chronic recurrent multifocal osteomyelitis, Churg-Strauss syndrome, Cogan's syndrome, Corticosteroid-induced osteoporosis, Costosternal syndrome, CREST syndrome, Cryoglobulinemia, Degenerative joint disease, Dermatomyositis, Diabetic finger sclerosis, Diffuse idiopathic skeletal hyperostosis (DISH), Discitis, Discoid lupus erythematosus, Drug-induced lupus, Duchenne's muscular dystrophy, Dupuytren's contracture, Ehlers-Danlos syndrome, Enteropathic arthritis, Epicondylitis, Erosive inflammatory osteoarthritis, Exercise-induced compartment syndrome, Fabry's disease, Familial Mediterranean fever, Farber's lipogranulomatosis, Felty's syndrome, Fibromyalgia, Fifth's disease, Flat feet, Foreign body synovitis, Freiberg's disease, Fungal arthritis, Gaucher's disease, Giant cell arthritis, Gonococcal arthritis, Goodpasture's syndrome, Gout, Granulomatous arthritis, Hemarthrosis, hemochromatosis, Henoch-Schonlein purpura, Hepatitis B surface antigen disease, Hip dysplasia, Hurler syndrome, Hypermobility syndrome, Hypersensitivity vasculitis, Hypertrophic osteoarthropathy, Immune complex disease, Impingement syndrome, Jaccoud's arthropathy, Juvenile ankylosing spondylitis, Juvenile dermatomyositis, Juvenile rheumatoid arthritis, Kawasaki disease, Kienbock's disease, Legg-Calve-Perthes disease, Lesch-Nyhan syndrome, Linear scleroderma, Lipoid dermatoarthritis, Lofgren's syndrome, Lyme disease, Malignant synovioma, Marfan's syndrome, Medial plica syndrome, Metastatic carcinomatous arthritis, Mixed connective tissue disease (MCTD), Mixed cryoglobulinemia, Mucopolysaccharidosis, Multicentric reticulohistiocytosis, Multiple epiphyseal dysplasia, Mycoplasmal arthritis, Myofascial pain syndrome, Neonatal lupus, Neuropathic arthropathy, Nodular panniculitis, Ochronosis, Olecranon bursitis, Osgood-Schlatter's disease, Osteoarthritis, Osteochondromatosis, Osteogenesis imperfecta, Osteomalacia, Osteomyelitis, Osteonecrosis, Osteoporosis, Overlap syndrome, Pachydermoperiostosis Paget's disease of bone, Palindromic rheumatism, Patellofemoral pain syndrome, Pellegrini-Stieda syndrome, Pigmented villonodular synovitis, Piriformis syndrome, Plantar fasciitis, Polyarteritis nodosa, Polymyalgia rheumatica, Polymyositis, Popliteal cysts, Posterior tibial tendonitis, Pott's disease, Prepatellar bursitis, Prosthetic joint infection, Pseudoxanthoma elasticum, Psoriatic arthritis, Raynaud's phenomenon, Reactive arthritis/Reiter's syndrome, Reflex sympathetic dystrophy syndrome, Relapsing polychondritis, Retrocalcaneal bursitis, Rheumatic fever, Rheumatoid arthritis, Rheumatoid vasculitis, Rotator cuff tendonitis, Sacroiliitis, Salmonella osteomyelitis, Sarcoidosis, Saturnine gout, Scheuermann's osteochondritis, Scleroderma, Septic arthritis, Seronegative arthritis, Shigella arthritis, Shoulder-hand syndrome, Sickle cell arthropathy, Sjogren's syndrome, Slipped capital femoral epiphysis, Spinal stenosis, Spondylolysis, Staphylococcus arthritis, Stickler syndrome, Sub-acute cutaneous lupus, Sweet's syndrome, Sydenham's chorea, Syphilitic arthritis, Systemic lupus erythematosus (SLE), Takayasu's arteritis, Tarsal tunnel syndrome, Tennis elbow, Tietse's syndrome, Transient osteoporosis, Traumatic arthritis, Trochanteric bursitis, Tuberculosis arthritis, Arthritis of Ulcerative colitis, Undifferentiated connective tissue syndrome (UCTS), Urticarial vasculitis, Viral arthritis, Wegener's granulomatosis, Whipple's disease, Wilson's disease and Yersinial arthritis which are characterized by (i) The induction of matrix metallo-proteinases by the pro inflammatory cytokines, (ii) Prostaglandin synthesis by the action of cyclo oxygenase enzymes, (iii) Leukotriene B4 synthesis by 5-lipoxygenase enzyme, and (iv) Breakdown of the connective tissue framework.
(D) A comprehensive prophylactic/therapeutic regimen that unexpectedly brings about an exponential decrease in the levels of pro inflammatory cytokines implicated in inflammatory arthritis.

Other features and advantages of the present invention will become apparent from the following more detailed description of the preferred embodiment, which illustrates, by way of example, the principle of the invention.

### DESCRIPTION OF THE FIGURES

FIG. 1 shows the mean decrease in VAS assessment of pain stiffness and swelling in patient groups where one group was treated with the composition of the present invention and the other with just glucosamine sulphate.

FIG. 2 shows the comparative mean decrease in Western Ontario McMaster Universities Osteoarthritis Index by using the composition of the present invention and Glucosamine sulphate.

### DETAILED DESCRIPTION OF THE MOST PREFERRED EMBODIMENT

In the most preferred embodiment, the present invention relates to sustained-release medicaments/drug compositions/formulations/therapeutics for the management of pain and inflammation associated disorders in general. More preferably, the sustained-release compositions of the present invention are effective for the prevention/treatment of arthritic inflammation. The sustained-release compositions comprise effective amounts of glucosamine, curcuminoids, boswellic acids and piperine (extract of Piper nigrum).

In another most preferred embodiment, the sustained release medicaments/drug compositions/formulations/therapeutics for the management of pain and inflammation comprise (i) from about 100 mg to about 8000 mg of glucosamine; (ii) from about 20 mg to about 8000 mg of curcuminoids; (iii) 20 mg-2000 mg of Boswellia serrata gum resin, its extractives, isolates or derivatives represented by one or more entities selected from the group consisting of beta-boswellic acid, acetyl-beta boswellic acid, 11-keto-boswellic acid, acetyl-11-keto-boswellic acid and their congeners or derivatives either as single entities or combinations thereof; and (iv) 1 mg to 5 mg of piperine. In another more preferred embodiment, the said sustained release medicaments/drug compositions/formulations/therapeutics comprise 20 mg-2000 mg of acetyl-11-keto-boswellic acid and their congeners or derivatives either as single entities or combinations thereof.

In another most preferred embodiment, the sustained-release the sustained release medicaments/drug compositions/formulations/therapeutics for the management of pain and inflammation is suited for oral ingestion. In an alternative embodiment, the orally ingestable sustained-release medicaments/drug compositions/formulations/therapeutics for the management of pain and inflammation also comprises 50 micrograms to 200 micrograms of bioavailable organic selenium nutritional supplement selected from the group consisting of selenomethionine, methylselenocysteine, derivatives of selenomethionine, derivatives of methylselenocysteine, isomeric peptides of selenomethionine, isomeric peptides of methylselenocysteine, high selenium yeast and selenium enriched vegetables. The actives including Curcuminoids, Boswellia serrata gum resin, its extractives, isolates or derivatives, Piper Nigrum extract and Glucosamine Sulphate are blended and entrapped into a retardant matrix of excipients to designate a sustained release for over a period of 12 hours. The matrix provides a barrier for the “erosion” of the actives out of the tablet. Since all four actives are required to be released equitably, the actives are blended uniformly. Hence this allows all the four to be eluted in unison. The principal advantage of a sustained-release formulation is that the dosing frequency is reduced.

The efficacy of the sustained-release formulation of the present invention comprising (i) from about 100 mg to about 8000 mg of glucosamine; (ii) from about 20 mg to about 8000 mg of curcuminoids; (iii) 20 mg-2000 mg of Boswellia serrata gum resin, its extractives, isolates or derivatives represented by one or more entities selected from the group consisting of beta-boswellic acid, acetyl-beta boswellic acid, 11-keto-boswellic acid, acetyl-11-keto-boswellic acid and their congeners or derivatives either as single entities or combinations thereof; and (iv) 1 mg to 5 mg of piperine in the therapeutic management of osteoarthritis of the knee is presented herein below as Example I.

### EXAMPLE I

### A Double Blind, Randomized Study Designed to Evaluate the Efficacy of the Sustained-Release Formulation of the Present Invention in Comparison to Glucosamine Sulphate Alone for the Treatment of Osteoarthritis of the Knee

Introduction

Osteoarthritis (OA) is a chronic, degenerative disorder of multifactorial aetiology, characterized by loss of articular cartilage and periarticular bone remodeling. OA causes joint pain, typically worse with weight bearing and activity, and stiffness after inactivity. There is no cure, and gradual, although slow, progression is most common (1).

Pharmacological treatment for OA can be divided into two groups: symptom-modifying drugs and disease modifying drugs. Symptom modifying drugs like simple analgesics, non-steroidal anti-inflammatory drugs (NSAIDS) are at present the prescription of choice and are effective in relieving symptoms of OA. However, they are also the cause of serious side effects (2).

On the other hand, disease modifying drugs like glucosamine sulphate have shown promising results with effect sizes ranging from moderate to high (3,4). It has been shown to regenerate cartilage and thus may be a potential treatment for degenerative joint disease like OA by limiting further degeneration and promoting tissue repair (5,6).

The ideal treatment of OA would be a combination of symptom and disease modifying drugs with minimum toxicity. The sustained-release herb-mineral formulation of the present invention aims to achieve this. It contains glucosamine sulphate with Boswellia serrata and curcuminoids. Boswellia serrata extract has good anti-inflammatory and analgesic activity coupled with good tolerability (7). Curcuminoids are antioxidants which could further help in relieving symptoms of osteoarthritis.

This double blind, randomized study was designed to evaluate the efficacy of the said herb-mineral formulation in comparison to using glucosamine sulphate alone for the treatment of osteoarthritis of the knee.

Materials and Methods

Selection of Patients

The institutional ethics committee approved the study protocol. Patients were recruited from the orthopaedic outpatient clinic of a tertiary care hospital. Consecutive outpatients of either sex, aged between 40-65 years in ARA functional class I, II or III and radiologically diagnosed to have osteoarthritis of the knee were identified. Clinically pain or discomfort should have been experienced in the affected knee on most days for the previous 3 months. Of these, patients already undergone knee arthroplasty, taken glucosamine in the previous 3 months or any intraarticular injection within previous month, having renal and/or hepatic disease, diabetes mellitus or a disabling co-morbidity were excluded.

Study Design, Assessments and Treatment

After giving their written informed consent, patients were assessed at baseline for demographic and clinical characteristics. They were then randomized to receive either glucosamine sulphate or a combination of glucosamine sulphate, Boswellia serrata and curcuminoids twice-daily in a double blind manner for a total period of 90 days. Both the medications were identical in appearance and taste. Treatment of associated disease was allowed at the discretion of the physician. Patients were assessed at baseline and at the end of 90 days. Four separate 10 cm visual analogue scales (VAS) were used to measure pain in affected knee at rest, on movement and the patients overall assessment of swelling and morning stiffness. VAS was scored from 0 to 10, where 0 indicates no pain/swelling/stiffness and 10 is the worst pain/swelling/stiffness imaginable.

The Western Ontario McMaster Universities Osteoarthritis Index (WOMAC) (8) was used to evaluate pain, stiffness and physical function of the knee. The pain and stiffness subscales of the WOMAC have scores of 0-20 and 0-8 respectively, with 0 representing no pain or stiffness. The functional subscale of the WOMAC produces a range of scores from 0 to 68, where 0 indicates no functional disability and 68 indicates extreme difficulty performing daily living tasks. Adherence to trial medication was recorded. Adverse events, patient withdrawals and concomitant illnesses were recorded in accordance with good clinical practice guidelines.

Statistical Analysis

The primary outcome measure was the change in VAS and WOMAC assessment of pain and stiffness in the affected knee. Response to treatment was analysed on an intention to treat basis, and the continuous variables were tested for significance by the standard error of difference in means. Categorical data were expressed as percentages with their 95% confidence intervals (CI). Significance was defined as a P value of less than 0.05.

Results

Baseline Analysis

A total of 50 consecutive eligible patients with osteoarthritis of the knee were randomized to either of the treatment groups. At baseline, patient characteristics were similar between the two treatment groups (Table 1). Most patients were in their sixth decade, with a history of osteoarthritis of almost 4 years. Majority of the patients were females and both groups were matched in their scores for different subscales of the WOMAC.

Efficacy Data

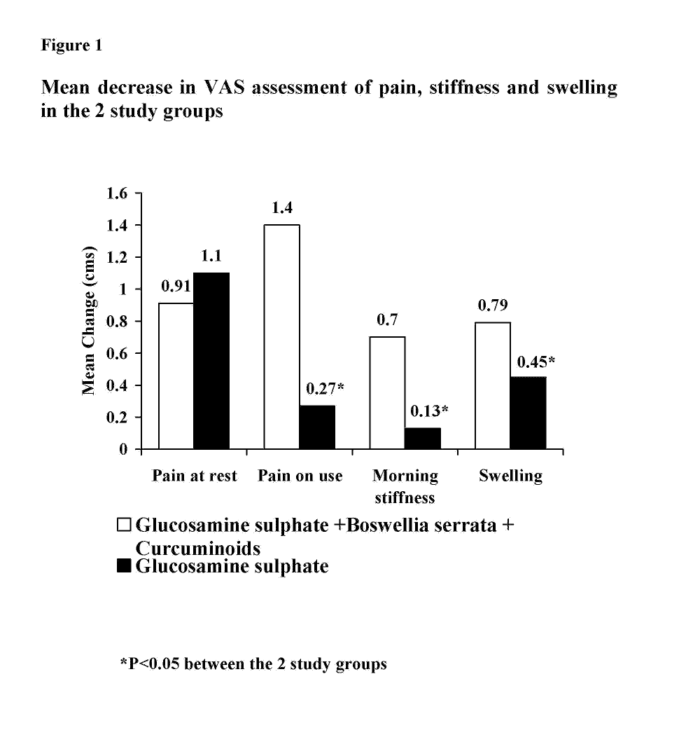

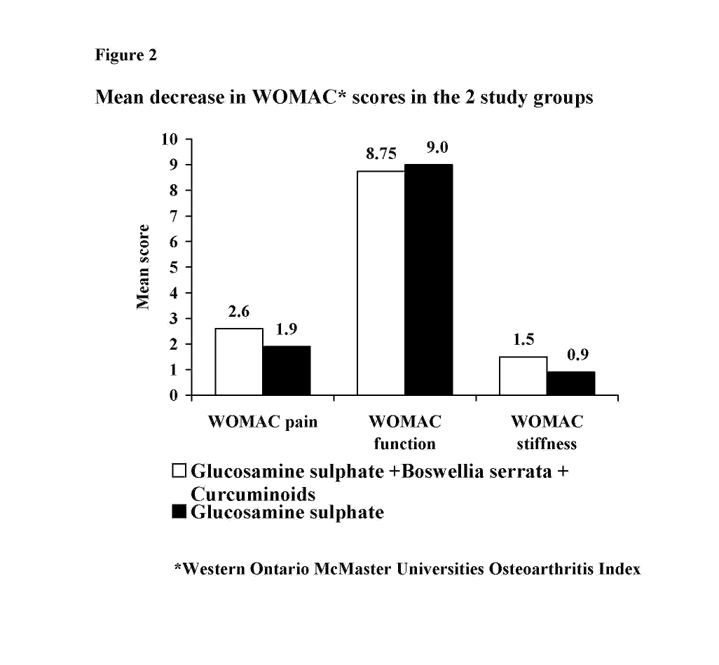

At the end of 3 months, patients showed a decrease both in VAS and WOMAC scores after the study treatments. The mean decrease in VAS and WOMAC scores for both the study groups is shown in FIG. 1 and FIG. 2 respectively. The trend in most of the scores was in favour of the herb-mineral formulation of glucosamine sulphate, Boswellia serrata and curcuminoid. There was a significant difference in favor of this combination for pain on use, morning stiffness and swelling (FIG. 1).

Mean (95% CI) VAS scores for pain on use decreased by 1.4 (1.04 to 1.78, P<0.001); for morning stiffness by 0.7 (0.41 to 0.99, P<0.001); for swelling by 0.79 (0.42 to 1.16, P<0.001) in patients on this herb-mineral formulation. In contrast for patients only on glucosamine sulphate, mean (95% CI) VAS scores for pain on use decreased by 0.27 (0.01 to 0.55, P=0.05); for morning stiffness by 0.13 (0.28 to 0.55, P=0.51); for swelling by 0.45 (0.03 to 0.88, P=0.04). The WOMAC score reduction also had a similar trend as the VAS score reduction (FIG. 2).

Adherence and Adverse Events

All 50 patients completed the study and there were no patients lost to follow up or withdrawn. Adherence to both treatment groups exceeded 80%. No serious adverse effects of treatment were reported during the trial. 3 (12.0%) patients in the glucosamine group and 1 (4.0%) patient in the herb-mineral formulation group reported increase in pain. Headache was reported by 4 (16.0%) and 1 (4.0%) patients in the glucosamine alone, and herb-mineral group respectively.

Discussion

In an outpatient tertiary care setting, the herb-mineral formulation containing glucosamine sulphate, Boswellia serrata and curcuminoids offered significant relief from pain and stiffness in patients with osteoarthritis as compared to glucosamine sulphate alone.

This study supports the findings of earlier reports which have suggested that glucosamine supplementation provides some degree of pain relief and improved mobility (9,10) and Boswellia serrata extract is well tolerated and has good anti-inflammatory property (7). It further demonstrated that combining a disease modifying and symptom modifying drug might be better than a disease modifying drug alone. The good tolerability of this new herb-mineral formulation is also an advantage in view of the long standing treatment of OA.

Interest in complementary therapies in many areas of medicine is growing and patients with chronic diseases, concerned about the possible adverse effects of many orthodox treatments, frequently seek alternatives that are seen as ‘natural’ and therefore harmless (11). Clinicians have a responsibility to offer rational, evidence-based advice about complementary therapies, but in many cases this is restricted by the lack of scientific evidence. This double blind randomized study has provided good scientific evidence of the safety and efficacy of this new formulation.

The study has limitations. The comparative efficacy of the two formulations was studied over the short term and possible benefits over a longer period need to be assessed. The benefits related to disease modifying properties especially need to be studied longer and confirmed in larger number of patients.

The results of this study suggest that in comparison to glucosamine sulphate alone, a combination of glucosamine sulphate, Boswellia serrata and curcuminoids is more effective in relieving pain and stiffness in patients with osteoarthritis of the knee (Figure I and Figure II). This new herb-mineral formulation could be a good combination of disease modifying and symptom modifying drugs for the treatment of osteoarthritis.

### EXAMPLE II

### Enzyme Linked Immuno Sorbent Assay (ELISA) for the Detection and Determining the Concentration of the Pro Inflammatory Biomarkers Ion in Plasma of Osteoarthritic Patients

Leukotriene B4 Assay

Principle of the Assay

This assay is based on the forward sequential competitive binding technique (Competitive ELISA method) in which LTB4 present in a sample competes with a fixed amount of horseradish peroxidase (HRP)-labeled LTB4 for sites on a chicken polyclonal antibody. During the incubations, the chicken polyclonal antibody becomes bound to the rabbit anti-chicken antibody coated onto the microplate. Following a wash to remove excess conjugate and unbound sample, a substrate solution is added to the wells to determine the bound enzyme activity. Immediately following color development, the absorbance is read at 450 nm. The intensity of the color is inversely proportional to the concentration of LTB4 in the sample.

The results (Table I) showed an exponential decrease in the levels of LTB4 in patients afflicted with osteoarthritis who were treated with the sustained-release composition of the present invention. The decrease was attributed to the synergism of the actives included in the said composition.

TABLE I

| | LTB4 levels in patients | LTB4 levels in patients |
|---|---|---|
| | afflicted with osteoarthritic | afflicted with osteoarthritic |
| | inflammation before treatment | inflammation after treatment |
| Patient | with the sustained-release | with the sustained-release |
| Sample | formulation of the present | formulation of the present |
| (n = 6) | invention (picograms/ml) | invention (picograms/ml) |
| 1. | 868.465500 | 105.4610 |
| 2. | 851.137800 | 352.8008 |
| 3. | 798.156700 | 333.7614 |
| 4. | 1098.901000 | 510.5987 |
| 5. | 942.185100 | 497.2155 |
| 6. | 551.708300 | 406.6279 |

Human Tumor Necrosis Factor—Alpha Assay

Principle of the Assay

This assay employs the quantitative sandwich enzyme immunoassay technique (direct ELISA method). A monoclonal antibody specific for TNF-alpha has been pre-coated onto a microplate. Standards and samples are pipetted into the wells and any TNF-_present is bound by the immobilized antibody. After washing away any unbound substances, an enzyme-linked polyclonal antibody specific for TNF-alpha is added to the wells. Following a wash to remove any unbound antibody-enzyme reagent, a substrate solution is added to the wells. After an incubation period, an amplifier solution is added to the wells and color develops in proportion to the amount of TNF-alpha bound in the initial step. The color development is stopped and the intensity of the color is measured.

Result (Table II) showed an exponential decrease in the levels of TNF-alpha in patients afflicted with osteoarthritis who were treated with the sustained-release composition of the present invention. The decrease was attributed to the synergism of the actives included in the said composition.

TABLE II

| | TNF-alpha levels in patients | TNF-alpha levels in patients |
|---|---|---|
| | afflicted with osteoarthritic | afflicted with osteoarthritic |
| | inflammation before treatment | inflammation after treatment |
| Patient | with the sustained-release | with the sustained-release |
| Sample | formulation of the present | formulation of the present |
| (n = 5) | invention (picograms/ml) | invention (picograms/ml) |
| 1. | 0.7518086 | 0.089 |
| 2. | 1.099271 | 0.097 |
| 3. | 0.5616834 | 0.096 |
| 4. | 1.219577 | 0.103 |
| 5. | 0.4743956 | 0.235 |

### REFERENCES

1. Grainger R, Cicuttini F M. Medical management of osteoarthritis of the knee and hip joints. MJA 2004; 180: 232-236.
2. Lequesne M, Brandt K, Bellamy N, Moskowitz R, Menkes C J, Pelletier J P, Altman R. Guidelines for testing slow acting drugs in osteoarthritis. J Rheumatol Suppl 1994; 41: 65-71; discussion 72-3.
3. McAlindon T E, La Valley M P, Gulin J P, Felson D T. Glucosamine and chondroitin for treatment of osteoarthritis: a systematic quality assessment and meta-analysis. JAMA 2000; 283: 1469-1475.
4. Towheed T E, Anastassiades T P, Shea B, Houpt J, Welch V, Hochberg M C. Glucosamine therapy for treating osteoarthritis. Cochrane Database Syst Rev 2001: CD002946.
5. Reichelt A, Forster K, Fisher M et al. Efficacy and safety of intramuscular glucosamine sulphate in osteoarthritis of the knee. A randomized, placebo-controlled double-blind study. Arzneimittel Forschung 1994; 44: 75-80.
6. Barclay T, Tsourounis C, McCart G. Glucosamine. Ann Pharmacother 1998; 32: 574-9.
7. Kimmatkar N, Thawani V, Hingorani L, Khiyani R. Efficacy and tolerability of Boswellia serrata extract in treatment of osteoarthritis of knee—a randomized double blind placebo controlled trial. Phytomedicine 2003 January; 10(1): 3-7.
8. Bellamy N, Buchanan W W, Goldsmith C H, Campbell J. Validation study of WOMAC: a health status instrument for measuring clinically important patient-relevant outcomes following total hip or knee arthroplasty in osteoarthritis. J Orthop Rheum 1988; 1: 95-108.
9. Pujalte J, Llavore E, Ylescupidez F. Double blind clinical evaluation of oral glucosamine sulphate in the basic treatment of osteoarthritis. Curr Med Res Opin 1980; 7: 110-114.
10. Vaz A. Double blind clinical evaluation of the relative efficacy of ibuprofen and glucosamine sulphate in the management of osteoarthritis of the knee in outpatients. Curr Med Res Opin 1982; 8: 145-9.
11. Hitchon C A, Cheang M, Wardell J, El-Gabalawy H S, Canvin J M G. Alternative therapy use in rheumatoid arthritis: prevalence and patient characteristics. Arthritis Rheum 1998; 41 (Suppl): S79.

We claim:

1. Sustained-release medicaments/drug compositions/formulations/therapeutics comprising a combination of symptom modifying and disease modifying agents for the prophylactic/therapeutic management of pain, inflammation and inflammation associated disorders.

2. Sustained-release medicaments/drug compositions/formulations/therapeutics according to claim 1, wherein the said medicaments/drug compositions/formulations/therapeutics comprise,
(i) From about 100 mg to about 8000 mg of glucosamine;
(ii) From about 20 mg to about 8000 mg of curcuminoids
(iii) From about 20 mg to about 2000 mg of Boswellia serrata gum resin, its extractives, isolates or derivatives represented by one or more entities selected from the group consisting of beta-boswellic acid, acetyl-beta boswellic acid, 11-keto-boswellic acid, acetyl-11-keto-beta-boswellic acid and their congeners or derivatives either as single entities or combinations thereof; and
(iv) From about 1 mg to about 5 mg of piperine.

3. Sustained-release medicaments/drug compositions/formulations/therapeutics according to claim 2, wherein glucosamine occurs as glucosamine sulphate.

4. Sustained-release medicaments/drug compositions/formulations/therapeutics according to claim 2, wherein the derivative of Boswellia serrata gum resin is acetyl-11-keto-beta-boswellic acid.

5. Sustained-release medicaments/drug compositions/formulations/therapeutics according to claim 1, wherein the inflammation associated disorder is inflammatory arthritis represented by the group consisting of Achilles tendonitis, Achondroplasia, Acromegalic arthropathy, Adhesive capsulitis, Adult onset Still's disease, Ankylosing spondylitis, Anserine bursitis, Avascular necrosis, Behcet's syndrome, Bicipital tendonitis, Blount's disease, Brucellar spondylitis, Bursitis, Calcaneal bursitis, Calcium pyrophosphate dihydrate (CPPD), Crystal deposition disease, Caplan's syndrome, Carpal tunnel syndrome, Chondrocalcinosis, Chondromalacia patellae, Chronic synovitis, Chronic recurrent multifocal osteomyelitis, Churg-Strauss syndrome, Cogan's syndrome, Corticosteroid-induced osteoporosis, Costosternal syndrome, CREST syndrome, Cryoglobulinemia, Degenerative joint disease, Dermatomyositis, Diabetic finger sclerosis, Diffuse idiopathic skeletal hyperostosis (DISH), Discitis, Discoid lupus erythematosus, Drug-induced lupus, Duchenne's muscular dystrophy, Dupuytren's contracture, Ehlers-Danlos syndrome, Enteropathic arthritis, Epicondylitis, Erosive inflammatory osteoarthritis, Exercise-induced compartment syndrome, Fabry's disease, Familial Mediterranean fever, Farber's lipogranulomatosis, Felty's syndrome, Fibromyalgia, Fifth's disease, Flat feet, Foreign body synovitis, Freiberg's disease, Fungal arthritis, Gaucher's disease, Giant cell arthritis, Gonococcal arthritis, Goodpasture's syndrome, Gout, Granulomatous arthritis, Hemarthrosis, hemochromatosis, Henoch-Schonlein purpura, Hepatitis B surface antigen disease, Hip dysplasia, Hurler syndrome, Hypermobility syndrome, Hypersensitivity vasculitis, Hypertrophic osteoarthropathy, Immune complex disease, Impingement syndrome, Jaccoud's arthropathy, Juvenile ankylosing spondylitis, Juvenile dermatomyositis, Juvenile rheumatoid arthritis, Kawasaki disease, Kienbock's disease, Legg-Calve-Perthes disease, Lesch-Nyhan syndrome, Linear scleroderma, Lipoid dermatoarthritis, Lofgren's syndrome, Lyme disease, Malignant synovioma, Marfan's syndrome, Medial plica syndrome, Metastatic carcinomatous arthritis, Mixed connective tissue disease (MCTD), Mixed cryoglobulinemia, Mucopolysaccharidosis, Multicentric reticulohistiocytosis, Multiple epiphyseal dysplasia, Mycoplasmal arthritis, Myofascial pain syndrome, Neonatal lupus, Neuropathic arthropathy, Nodular panniculitis, Ochronosis, Olecranon bursitis, Osgood-Schlatter's disease, Osteoarthritis, Osteochondromatosis, Osteogenesis imperfecta, Osteomalacia, Osteomyelitis, Osteonecrosis, Osteoporosis, Overlap syndrome, Pachydermoperiostosis Paget's disease of bone, Palindromic rheumatism, Patellofemoral pain syndrome, Pellegrini-Stieda syndrome, Pigmented villonodular synovitis, Piriformis syndrome, Plantar fasciitis, Polyarteritis nodosa, Polymyalgia rheumatica, Polymyositis, Popliteal cysts, Posterior tibial tendonitis, Pott's disease, Prepatellar bursitis, Prosthetic joint infection, Pseudoxanthoma elasticum, Psoriatic arthritis, Raynaud's phenomenon, Reactive arthritis/Reiter's syndrome, Reflex sympathetic dystrophy syndrome, Relapsing polychondritis, Retrocalcaneal bursitis, Rheumatic fever, Rheumatoid arthritis, Rheumatoid vasculitis, Rotator cuff tendonitis, Sacroiliitis, Salmonella osteomyelitis, Sarcoidosis, Saturnine gout, Scheuermann's osteochondritis, Scleroderma, Septic arthritis, Seronegative arthritis, Shigella arthritis, Shoulder-hand syndrome, Sickle cell arthropathy, Sjogren's syndrome, Slipped capital femoral epiphysis, Spinal stenosis, Spondylolysis, Staphylococcus arthritis, Stickler syndrome, Sub-acute cutaneous lupus, Sweet's syndrome, Sydenham's chorea, Syphilitic arthritis, Systemic lupus erythematosus (SLE), Takayasu's arteritis, Tarsal tunnel syndrome, Tennis elbow, Tietse's syndrome, Transient osteoporosis, Traumatic arthritis, Trochanteric bursitis, Tuberculosis arthritis, Arthritis of Ulcerative colitis, Undifferentiated connective tissue syndrome (UCTS), Urticarial vasculitis, Viral arthritis, Wegener's granulomatosis, Whipple's disease, Wilson's disease and Yersinial arthritis.

6. Sustained-release medicaments/drug compositions/formulations/therapeutics according to claim 1, wherein the symptom modifying agent inhibits the induction of pro inflammatory cytokines.

7. Sustained-release medicaments/drug compositions/formulations/therapeutics according to claim 1, wherein the symptom modifying agent inhibits the induction of leukotriene B4.

8. Sustained-release medicaments/drug compositions/formulations/therapeutics according to claim 1, wherein the symptom modifying agent inhibits the synthesis of prostaglandins.

9. Sustained-release medicaments/drug compositions/formulations/therapeutics according to claim 1, wherein the symptom modifying agent inhibits the activity of matrix metalloproteinases.

10. Sustained-release medicaments/drug compositions/formulations/therapeutics according to claim 1, wherein the disease modifying agent helps in the repair of damaged connective tissue as a result of the inflammatory process.

11. A method of prophylactic/therapeutic management of pain, inflammation and inflammation associated disorder, said method comprising the oral administration to a subject in need of such management, sustained-release medicaments/drug compositions/formulations/therapeutics comprising a combination of symptom modifying agents and disease modifying agents.

12. The method of prophylactic/therapeutic management of pain, inflammation and inflammation associated disorder according to claim 11, said method comprising the oral administration to a subject in need of such management, sustained-release medicaments/drug compositions/formulations/therapeutics comprising,
(i) From about 100 mg to about 8000 mg of glucosamine;
(ii) From about 20 mg to about 8000 mg of curcuminoids
(iii) From about 20 mg to about 2000 mg of Boswellia serrata gum resin, its extractives, isolates or derivatives represented by one or more entities selected from the group consisting of beta-boswellic acid, acetyl-beta boswellic acid, 11-keto-boswellic acid, acetyl-11-keto-beta-boswellic acid and their congeners or derivatives either as single entities or combinations thereof; and
(iv) From about 1 mg to about 5 mg of piperine.

13. The method according to claim 12, wherein glucosamine occurs as glucosamine sulphate.

14. The method according to claim 12, wherein the derivative of Boswellia serrata gum resin is acetyl-11-keto-beta-boswellic acid.

15. The method according to claim 11, wherein the inflammation associated disorder is inflammatory arthritis represented by the group consisting of Achilles tendonitis, Achondroplasia, Acromegalic arthropathy, Adhesive capsulitis, Adult onset Still's disease, Ankylosing spondylitis, Anserine bursitis, Avascular necrosis, Behcet's syndrome, Bicipital tendonitis, Blount's disease, Brucellar spondylitis, Bursitis, Calcaneal bursitis, Calcium pyrophosphate dihydrate (CPPD), Crystal deposition disease, Caplan's syndrome, Carpal tunnel syndrome, Chondrocalcinosis, Chondromalacia patellae, Chronic synovitis, Chronic recurrent multifocal osteomyelitis, Churg-Strauss syndrome, Cogan's syndrome, Corticosteroid-induced osteoporosis, Costosternal syndrome, CREST syndrome, Cryoglobulinemia, Degenerative joint disease, Dermatomyositis, Diabetic finger sclerosis, Diffuse idiopathic skeletal hyperostosis (DISH), Discitis, Discoid lupus erythematosus, Drug-induced lupus, Duchenne's muscular dystrophy, Dupuytren's contracture, Ehlers-Danlos syndrome, Enteropathic arthritis, Epicondylitis, Erosive inflammatory osteoarthritis, Exercise-induced compartment syndrome, Fabry's disease, Familial Mediterranean fever, Farber's lipogranulomatosis, Felty's syndrome, Fibromyalgia, Fifth's disease, Flat feet, Foreign body synovitis, Freiberg's disease, Fungal arthritis, Gaucher's disease, Giant cell arthritis, Gonococcal arthritis, Goodpasture's syndrome, Gout, Granulomatous arthritis, Hemarthrosis, hemochromatosis, Henoch-Schonlein purpura, Hepatitis B surface antigen disease, Hip dysplasia, Hurler syndrome, Hypermobility syndrome, Hypersensitivity vasculitis, Hypertrophic osteoarthropathy, Immune complex disease, Impingement syndrome, Jaccoud's arthropathy, Juvenile ankylosing spondylitis, Juvenile dermatomyositis, Juvenile rheumatoid arthritis, Kawasaki disease, Kienbock's disease, Legg-Calve-Perthes disease, Lesch-Nyhan syndrome, Linear scleroderma, Lipoid dermatoarthritis, Lofgren's syndrome, Lyme disease, Malignant synovioma, Marfan's syndrome, Medial plica syndrome, Metastatic carcinomatous arthritis, Mixed connective tissue disease (MCTD), Mixed cryoglobulinemia, Mucopolysaccharidosis, Multicentric reticulohistiocytosis, Multiple epiphyseal dysplasia, Mycoplasmal arthritis, Myofascial pain syndrome, Neonatal lupus, Neuropathic arthropathy, Nodular panniculitis, Ochronosis, Olecranon bursitis, Osgood-Schlatter's disease, Osteoarthritis, Osteochondromatosis, Osteogenesis imperfecta, Osteomalacia, Osteomyelitis, Osteonecrosis, Osteoporosis, Overlap syndrome, Pachydermoperiostosis Paget's disease of bone, Palindromic rheumatism, Patellofemoral pain syndrome, Pellegrini-Stieda syndrome, Pigmented villonodular synovitis, Piriformis syndrome, Plantar fasciitis, Polyarteritis nodosa, Polymyalgia rheumatica, Polymyositis, Popliteal cysts, Posterior tibial tendonitis, Pott's disease, Prepatellar bursitis, Prosthetic joint infection, Pseudoxanthoma elasticum, Psoriatic arthritis, Raynaud's phenomenon, Reactive arthritis/Reiter's syndrome, Reflex sympathetic dystrophy syndrome, Relapsing polychondritis, Retrocalcaneal bursitis, Rheumatic fever, Rheumatoid arthritis, Rheumatoid vasculitis, Rotator cuff tendonitis, Sacroiliitis, Salmonella osteomyelitis, Sarcoidosis, Saturnine gout, Scheuermann's osteochondritis, Scleroderma, Septic arthritis, Seronegative arthritis, Shigella arthritis, Shoulder-hand syndrome, Sickle cell arthropathy, Sjogren's syndrome, Slipped capital femoral epiphysis, Spinal stenosis, Spondylolysis, Staphylococcus arthritis, Stickler syndrome, Sub-acute cutaneous lupus, Sweet's syndrome, Sydenham's chorea, Syphilitic arthritis, Systemic lupus erythematosus (SLE), Takayasu's arteritis, Tarsal tunnel syndrome, Tennis elbow, Tietse's syndrome, Transient osteoporosis, Traumatic arthritis, Trochanteric bursitis, Tuberculosis arthritis, Arthritis of Ulcerative colitis, Undifferentiated connective tissue syndrome (UCTS), Urticarial vasculitis, Viral arthritis, Wegener's granulomatosis, Whipple's disease, Wilson's disease and Yersinial arthritis.

16. The method according to claim 11, wherein the symptom modifying agent inhibits the induction of pro inflammatory cytokines.

17. The method according to claim 11, wherein the symptom modifying agent inhibits the induction of leukotriene B4.

18. The method according to claim 11, wherein the symptom modifying agent inhibits the synthesis of prostaglandins.

19. The method according to claim 11, wherein the symptom modifying agent inhibits the activity of matrix metalloproteinases.

20. The method according to claim 11, wherein the disease modifying agent helps in the repair of connective tissue damage occurring as a result of the inflammatory process.

21. The method as in one of claims 11 or 12, in which the said method causes an exponential decrease in the induction/activity of pro inflammatory cytokines, leukotriene B4, prostaglandins and matrix metalloproteinases through synergistic effects mediated by the actives included therein.

22. Sustained-release medicaments/drug compositions/formulations/therapeutics as in one of claims 1 or 2, in which the said medicaments/drug compositions/formulations/therapeutics cause an exponential decrease in the induction/activity of pro inflammatory cytokines, leukotriene B4, prostaglandins and matrix metalloproteinases through synergistic effects mediated by the actives included therein.
